(19) United States
(12) Patent Application Publication
(10) Pub. No.: US 2007/0156043 A9
(43) Pub. Date: Jul. 5, 2007

(54) Method for obtaining magnetic resonance data representing movement in a subject

(76) Inventors: Ralf Loffler, Cordova, TN (US); Alistair Young, Auckland (NZ)

Correspondence Address: SCHIFF HARDIN, LLP;PATENT DEPARTMENT, 6600 SEARS TOWER, CHICAGO, IL, 60606-6473, US

(21) Appl. No.: 11/325,947

(22) Filed: Jan. 5, 2006

(30) Foreign Application Priority Data
Jan. 5, 2005 (DE) 10 2005 000 849.6

(51) Int. Cl. A61B 5/05

(52) U.S. Cl. 600/410

(57) ABSTRACT

In a method for acquisition of movement of a subject by magnetic resonance imaging, a pulse sequence is applied that spatially modulates the magnetization of the subject in one spatial direction, a spectral maximum of the image data in the Fourier domain representing the spatially-modulated subject is acquired by a three-dimensional imaging sequence with volume excitation of the nuclear spins, the acquired spectral maximum is Fourier transformed, and the movement of the subject is determined from the Fourier-transformed signal.

### BACKGROUND OF THE INVENTION

1. Field of the Invention

The present invention concerns a method for obtaining magnetic resonance data from a region of an examination subject exhibiting movement. The invention is in particular suitable for measurement of myocardial perfusion.

2. Description of the Prior Art

Magnetic resonance tomography used frequently clinically in everyday life for clarification of specific medical questions. Various developments have broadened the scope of usage of magnetic resonance tomography to many different questions, among other things the measurement of perfusion.

The measurement signals that are used for the image reconstruction and that enable a conclusion about processes in the examination subject follows exist mathematically in Fourier space or k-space. The MR image necessary for the imaging is obtained by Fourier transformation of k-space or the Fourier domain. To acquire an MR image, k-space or Fourier space is increasingly filled with raw data with the gradient strength and time being varied.

Furthermore, MR techniques have been developed with which advanced conclusions about the movement of organs (and therewith also about the perfusion in the heart) have become possible. One technique that is used is based on the modulation of the magnetization in the direction of the magnetic primary field by the application of various radio-frequency pulses that rotate the magnetization in a plane transverse to the longitudinal magnetization. One of these techniques is SPAMM (Spatial Modulation of Magnetization) that, among other things, is described in U.S. Pat. No. 5,054,489 and Axel et al.: “MR Imaging of Motion with Spatial Modulation of Magnetization”, Radiology, 171:841-845, 1989 and Axel et al.: “Heart Wall Motion: Improved Method of Spatial Modulation of Magnetization for MR Imaging”, Radiology, 172(1):349-350, 1989. The magnetization is spatially saturated by marking or tag pulses. In the imaging sequence that follows, intensity fluctuations or stripes bands with less signal intensity result in the MR image. A grid structure is obtained in the MR image by the application of these tag pulses in two different spatial directions. The movement of this grid structure over time can be used in order to make conclusions about the perfusion of the examined tissue. The perfusion is detected by establishing areas in which the heart moves only slightly or does not move at all. Conversely, the heart must then be less perfused in these regions. Examples for generation of marking patterns are described in U.S. Pat. No. 6,597,935. The body to be examined is then examined with an imaging sequence after the marking pulses.

In addition to the actual primary maximum in the center of Fourier space, secondary maxima result due to the use of the marking pulses before the actual imaging sequence.

Furthermore, for examination of the movement of the subject techniques have been developed that use the induced stripe pattern in order to acquire information about the perfusion. Techniques such as HARP/DENSE are based on the fact that the ancillary maxima present in Fourier space contain information about the movement. For example, for this purpose an image is reconstructed in which only the part of k-space or Fourier space in which the secondary maximum lies is considered. The image constructed in this manner is free of the anatomy forming the basis of the image and shows only the stripe pattem, so that by suitable post-processing techniques conclusions can be made about the perfusion from the movement of the stripe pattern.

In examinations, imaging sequences often must be used that require the data to be acquired under a breath-hold technique since respiratory movement would otherwise preclude a conclusion about the examined tissue. The subjects must repeatedly hold his or her breath for a relatively long time in the cramped MR acquisition space. For some patients this is not possible due to an unstable health state.

For this reason it is desirable to further shorten the acquisition time of the MR image.

U.S. Pat. No. 6,597,935 mentioned above describes a method in which the acquisition time in the perfusion imaging is shortened by controlling the imaging sequence such that the secondary maximum is acquired with a two-dimensional imaging sequence.

In “High speed 3D CSPAMM” in Proc. Intl. Soc. Mag. Reson. Med., 2004, p. 657, S. Ryf et al. describes a method to further shorten the acquisition time. The examined tissue is modulated with a three-dimensional marking sequence. The multiple secondary maxima resulting from this three-dimensional marking sequence are subsequently acquired with a three-dimensional imaging sequence. Furthermore, a relatively large range of Fourier space is acquired due to the acquisition of the multiple secondary maxima, which has a negative effect on the acquisition time.

### SUMMARY OF THE INVENTION

An object of the present invention is to further shorten the acquisition time for such imaging sequences while not impairing the signal/noise ratio.

This object is achieved by a method for acquisition of movements of a subject by magnetic resonance as follows.

A pulse sequence is first applied that spatially modulates the magnetization of the subject in one spatial direction. A spectral maximum of Fourier space of the spatially-modulated subject is subsequently acquired by a three-dimensional imaging sequence with a volume excitation of the nuclear spins. Due to the modulation of the magnetization, preferably the longitudinal magnetization along the primary axis of the static magnetic field B₀, two secondary maxima symmetrical to the primary maximum result in the Fourier domain. One of these secondary maxima is acquired with a three-dimensional imaging sequence. In the present invention, three-dimensional imaging sequence means that the entire volume is excited and the spatial resolution in the third dimension is achieved by an additional phase coding gradient. A 3D data volume can also be achieved when a plurality of different slices with a certain slice thickness are acquired in succession. However, the resolution in slice thickness is typically 10 times worse than in the plane of the two-dimensional imaging. In the present invention, a two-dimensional imaging sequence does not mean the acquisition of individual slices in succession; rather, an entire volume is excited by the RF pulse, and the spatial resolution subsequently ensues in all three dimensions. The Fourier transformation of the acquired spectral maximum is subsequently calculated and conclusions about the movement of the subject are made from the Fourier transformation. Due to the modulation of the magnetization in a single spatial direction, only two symmetrical maxima result in the Fourier domain. One of these maxima is then acquired with a three-dimensional imaging sequence (for example a gradient-echo sequence). Due to the acquisition of the three-dimensional space around the secondary maximum, this is optimally acquired such that the resulting stripe pattern can be further processed with a good signal/noise ratio. Due to the fact that only one maximum is acquired, the Fourier space to be acquired (and thereby the switching of the gradients) is optimized so that only a small volume must be acquired in Fourier space. This means that the acquisition time of the MR image is shortened. Relative to the method of U.S. Pat. No. 6,597,935, the present method has the advantage that the space around the ancillary maximum is acquired three-dimensionally and not two-dimensionally, such that the signal/noise ratio is better in the present method. Relative to the method described by S. Ryf, the present invention has the advantage that only a single maximum rather than multiple maxima must be acquired with the three-dimensional imaging sequence. The acquisition time can be decisively shortened, such that patients with poorer breath-hold capabilities can be examined.

According to a preferred embodiment, in the three-dimensional imaging sequence the Fourier space is acquired in the form of a solid cylinder around the spectral maximum. The spherically-symmetrical secondary maximum that is acquired by the three-dimensional imaging sequence is, at best, read out in a circle shape in the plane. For switching reasons, in the third spatial direction it is simplest to select a constant expansion such that Fourier space is ultimately read out in a cylindrical shape. The spectral maximum preferably lies on the longitudinal axis of the cylinder, such that the spectral maximum (and with it the information contained in the secondary maximum) can be acquired in the best possible manner. In the imaging sequence, the signal readout preferably ensues in the direction of the longitudinal axis of the cylinder since, as mentioned above, this can be realized most easily for switching reasons. Naturally, a different geometric shape can be selected (for example in the shape of a sphere), but the time of the signal readout would then have to be spherically varied in the signal readout direction.

The longitudinal magnetization preferably is modulated given the spatial modulation of the magnetization, as is possible (among other things) with the SPAMM technique. These techniques for spatially-targeted variation of the longitudinal magnetization are known to those skilled in the art and need not be described in detail herein.

Should the movement (in particular the perfusion in multiple spatial directions) be acquired, given the one-dimensional modulation of the magnetization and the subsequent three-dimensional signal readout the magnetization is modulated in one of the two other spatial directions perpendicular to the first spatial direction, and the other steps are subsequently repeated. For a three-dimensional acquisition, the magnetization of the third remaining spatial direction is ultimately modulated perpendicular to the first two spatial directions and the remaining steps (i.e. the acquisition of the spectral maximum with a three-dimensional imaging sequence, the calculation of the Fourier transformation and the determination of the movement from the Fourier-transformed signal) are then repeated. Relative to the prior art, each spatial direction is consequently individually modulated and the resulting ancillary maximum is three-dimensionally acquired for each spatial direction. The movement (in particular the perfusion) in the heart can be advantageously acquired with a better signal/noise ratio and in a shorter acquisition time.

### DESCRIPTION OF THE DRAWINGS

FIG. 1 shows an example of an intensity pattern for a one-dimensional marking pulse in Fourier space.

FIG. 2 illustrates the three-dimensional acquisition of this secondary maximum.

FIG. 3 is a flow chart for the individual steps for calculation of the perfusion.

### DESCRIPTION OF THE PREFERRED EMBODIMENTS

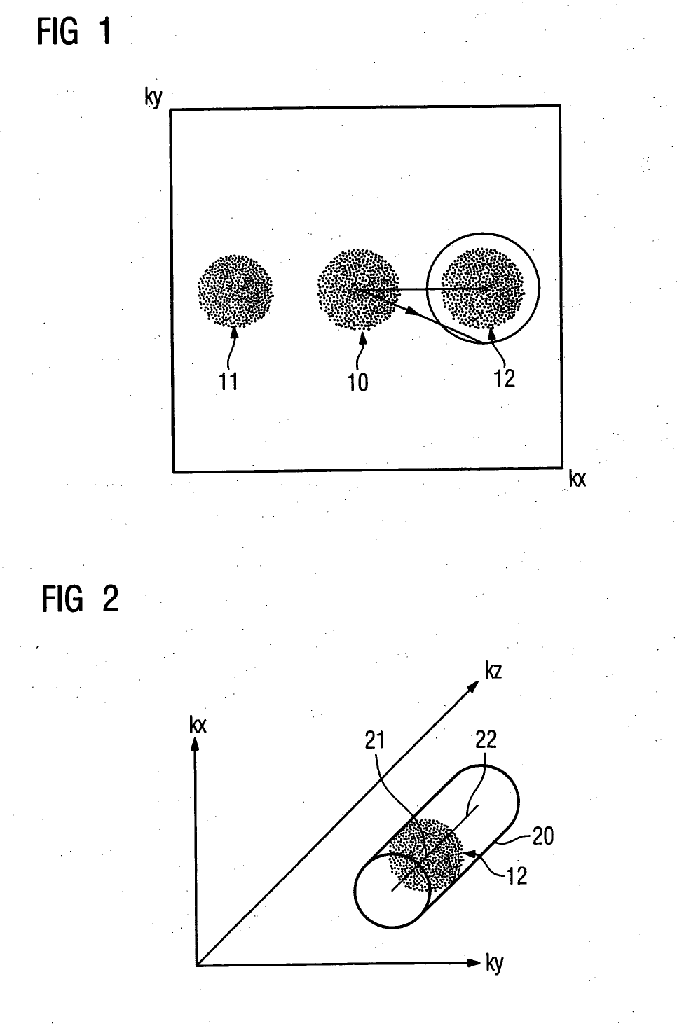

FIG. 1 shows k-space or the Fourier domain with a measurement signal after the longitudinal magnetization has been one-dimensionally modulated. In addition to the primary maximum 10, two secondary maxima 11 and 12 arise to the left and right of this due to the modulation of the magnetization. The positions of the secondary maxima result from the sequence of the pulses for modulation of the magnetization. For example, these secondary maxima lie further outward in Fourier space the higher the frequency of the magnetization pulses. To summarize, in addition to the primary maximum that contains the primary information of the MR image to be reconstructed, the position of the secondary maxima is precisely determined by the magnetization pulse sequence. The modulation of the magnetization by the SPAMM technique or CSPAMM technique is known to those skilled in the art and need not be explained in detail herein. In order to now obtain information about the movement of the examined subject, i.e. in particular the perfusion of the subject, the information contained in the secondary maximum is now used in order, for example, to place a grid in the perfused subject (for example the heart). From the temporal sequence of the grid movement, the movement of the grid can be tracked by acquisition of multiple images during the movement cycle, for example of the heart cycle. This grid movement now provides information about the perfusion of the examined subject. One field of application of this technique is, for example, in the determination of the perfusion of the myocardium. These techniques (used as HARP and DENSE) are known to those skilled in the art.

The secondary maximum 12 should now be acquired in the present example.

It is an important aspect of the present invention that the magnetization is only modulated in one spatial direction such that only two ancillary maxima result. Namely, when the magnetization is modulated in more than one spatial direction, a number of such secondary maxima 11 and 12 result, such that a number of secondary maxima would have to be acquired for information about the perfusion, which would undesirably lengthen the acquisition time.

In FIG. 2 it is shown how the ancillary maximum 12 is preferably acquired during the three-dimensional imaging sequence. The ancillary maximum 12 has an essentially radially-symmetrical expansion such that the space around the maximum is read out in a circular shape in the X/Y direction of k-space. The gradients in the x- or y-direction are the phase coding directions as well as the 3D direction. The signal readout preferably ensues constant in the k_{z}-direction, such that k-space specifying the maximum 12 is read out via the cylinder 20.

Other k-space acquisition techniques are naturally also possible, but it should be noted that the center of gravity in k-space 21 coincides with the harmonic maximum 12. A constant readout time is simple to effect, but other acquisition schemata such as, for example, a radial or spherical acquisition of k-space are also possible. If the maximum 12 is acquired cylindrically as shown in FIG. 2, the maximum should lie on the longitudinal axis 22 in the center of gravity of the cylinder.

As can be seen in FIG. 2, only a small volume around the maximum 12 must be read out, so that very small image acquisition times are possible, and an optimal signal/noise ratio is achieved by the three-dimensional acquisition of k-space around the maximum.

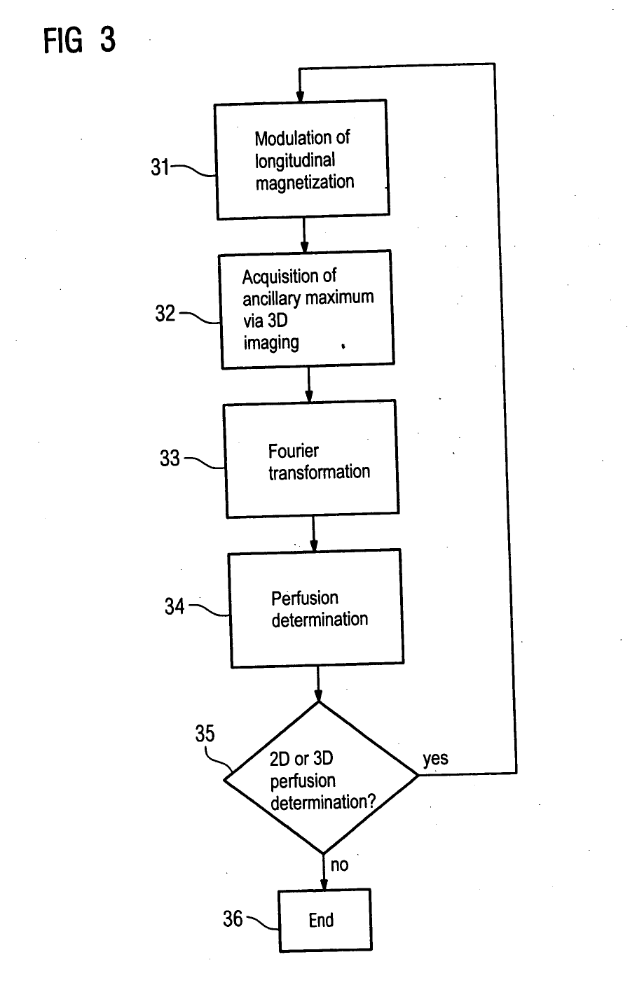

The various steps that are necessary for the measurement of the myocardium perfusion or for any other perfusion measurement are shown in FIG. 3.

The longitudinal magnetization in one spatial direction is modulated in a first step 31, for example via the SPAMM or CSPAMM technique. The position of an ancillary maximum can be definitely calculated with the magnetization pulses used in step 31, this secondary maximum being subsequently acquired in step 32 with a three-dimensional imaging sequence. This three-dimensional imaging sequence, for example, can be a gradient echo sequence or an EPI (echo planar imaging) sequence. Any other imaging sequence is possible with which images of the examination subject can be three-dimensionally acquired in short acquisition times, the imaging sequence also depending on the examined subject. As shown in FIG. 2, the secondary maximum is acquired via a cylindrical sampling [scanning] of Fourier space. The acquired data are subsequently Fourier-transformed in step 33, so a three-dimensional Fourier transformation is necessary due to the three-dimensional imaging. The perfusion is subsequently determined in step 34 from the Fourier-transformed data. For example, among other things a phase image can be calculated in step 34. The method for calculation of the perfusion from the acquired ancillary maxima are known to those skilled in the art and correspond to the methods known in the prior art (for example HARP and DENSE).

In step 35 it is queried whether two-dimensional or three-dimensional information about the perfusion should be acquired. If this is not the case, the method ends in step 36. If two-dimensional or three-dimensional perfusion information is desired, the steps 31 through 34 are repeated. For two-dimensional information, the magnetization is modulated in a direction perpendicular to the first spatial direction; the steps 32 through 34 are subsequently repeated. For a three-dimensional perfusion determination, the magnetization is subsequently modulated in the third spatial direction perpendicular to the first two spatial directions and the steps 32 through 34 are subsequently repeated in turn, whereby Fourier space is again read out in the form of a solid cylinder.

According to the invention, the associated secondary maximum is acquired in one spatial direction via a three-dimensional imaging sequence after the magnetization modulation. A good signal/noise ratio can be achieved in a short image acquisition time. This is subsequently repeated for both other spatial directions. Overall the perfusion can be determined in short image acquisition times with good signal/noise ratio with the method according to the invention.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for obtaining magnetic resonance data from a region of a subject exhibiting movement, comprising the steps of:
applying a pulse sequence at least to a region of an examination subject exhibiting movement, and with said pulse sequence spatially modulating magnetization in said region in at least one spatial direction;
applying a three-dimensional magnetic resonance imaging sequence with volume excitation of nuclear spins in said region to obtain magnetic resonance data represented in the Fourier domain exhibiting a spectral maximum in the Fourier domain;
automatically electronically Fourier transforming said spectral maximum to obtain a Fourier-transformed signal; and
automatically electronically determining movement in said region from said Fourier-transformed signal.

2. A method as claimed in claim 1 comprising obtaining and representing said magnetic resonance image data in the Fourier domain as a solid cylinder around said spectral maximum in said three-dimensional imaging sequence.

3. A method as claimed in claim 2 wherein said cylinder has a longitudinal axis, and acquiring and representing said magnetic resonance image data with said spectral maximum on said longitudinal axis of said cylinder.

4. A method as claimed in claim 3 comprising, in said three-dimensional imaging sequence, reading out said magnetic resonance image data along a direction of said longitudinal axis of said cylinder.

5. A method as claimed in claim 4 comprising, in said pulse sequence that spatially modulates the magnetization in said region in at least one spatial direction, modulating magnetization along said direction of said longitudinal axis of said cylinder.

6. A method as claimed in claim 1 wherein the step of applying a pulse sequence to spatially modulate magnetization in said region in at least one spatial direction comprises applying a pulse sequence that spatially modulates magnetization in said region in at least two spatial directions, and comprising obtaining magnetic resonance imaging data exhibiting a spectral maximum, Fourier transforming the spectral maximum to obtain a Fourier-transformed signal, and determining movement in said region from the Fourier transformed signal, for each of said spatial directions.

7. A method as claimed in claim 6 wherein the step of applying a pulse sequence to spatially modulate magnetization of the region in at least two spatial directions comprises applying a pulse sequence that spatially modulates magnetization in said region in three spatial directions.

8. A method as claimed in claim 1 wherein the step of automatically determining movement in said region from the Fourier-transformed signal comprises automatically electronically calculating phase images from the Fourier-transformed signal.
